(19) United States
(12) Patent Application Publication
(10) Pub. No.: US 2014/0017732 A1
(43) Pub. Date: Jan. 16, 2014

(54) TWO-LOOP DILUTE PREPROCESSING AND PRETREATMENT OF CELLULOSIC FEEDSTOCKS

(71) Applicant: Edeniq, Inc., (US)

(72) Inventors: Daniel A. Lane, Visalia, CA (US); James Kacmar, Visalia, CA (US)

(73) Assignee: Edeniq, Inc., Visalia, CA (US)

(21) Appl. No.: 13/840,950

(22) Filed: Mar. 15, 2013

Related U.S. Application Data
(60) Provisional application No. 61/671,303, filed on Jul. 13, 2012.

(51) Int. Cl. C12P 19/14; C12P 7/14

(52) U.S. Cl. CPC C12P 19/14; C12P 7/14
USPC 435/99; 435/162; 536/123.12; 536/123

(57) ABSTRACT

The present application describes a dilute two-loop method for treating biomass in the production of biofuels such as ethanol. The method involves washing the biomass to dissolve sugars that are associated with the biomass, and separating the washed biomass into a solids phase and a liquids phase. The solids phase is pretreated to render the biomass more susceptible to hydrolysis under conditions that do not produce substantial amounts of sugars. The pretreated biomass is separated into a second solids phase and second liquid phase, and the second solids phase is saccharified and fermented. The first and second liquid phases are recycled to dilute the biomass at various stages of the process.

### CROSS-REFERENCE TO RELATED PATENT APPLICATIONS

The present patent application claims benefit of priority to U.S. Patent Application No. 61/671,303, filed Jul. 13, 2012, which is incorporated by reference.

### BACKGROUND OF THE INVENTION

The concentration of sugars produced via enzymatic hydrolysis of pretreated biomass has historically been limited by the concentration of solids in the biomass slurry. Technology advances to improve sugars concentration, and subsequently ethanol concentration downstream, have centered on increasing solids loading to the pretreatment system. With higher solids loading comes increased wear on equipment and negative changes in rheological properties.

Most biomass has some ‘free sugars’ available with a hot water extraction, with the rest having to be made available by pretreatment and enzymes. However, pretreatment can destroy these free sugars and often leads to the formation of inhibitors to downstream biological activities. Examples of inhibitors include furfural, 5-hydroxymethylfurfural (5-HMF), and acetic acid.

In general, the problem is that the capital/process benefits of higher solids loadings are rapidly offset by higher inhibitor formation and concentration, requiring complex separation processes to remove the inhibitors, or dilution to reduce their impact.

The present application provides methods for processing biomass to ethanol and other valuable products that provide higher solids in the fermentation step, but with lower wear of equipment. The methods can also decrease the amounts of at least some inhibitors of fermentation and provide higher quantities of sugars to be used in fermentation to ethanol and other valuable products.

### BRIEF SUMMARY OF THE INVENTION

The present application provides a method of processing biomass, the method comprising:
(a) contacting the biomass with a first aqueous liquid under conditions sufficient to dissolve water-soluble sugars associated with the biomass;
(b) separating the first aqueous liquid from the biomass to produce a first liquid phase comprising the water-soluble sugars and a first solids phase comprising biomass solids;
(c) contacting the first solids phase with a second aqueous liquid under conditions sufficient to render the biomass more susceptible to hydrolysis but not releasing substantial amounts of sugars, thereby producing a pretreated biomass;
(d) separating the second aqueous liquid from the pretreated biomass to produce a second liquid phase and a second solids phase; and
(e) subjecting the second solids phase to conditions sufficient to produce fermentable sugars.

In some embodiments, the method further comprises fermenting the fermentable sugars.

In some embodiments, the method comprises treating the pretreated biomass to produce particles having a relatively uniform particle size. For example, the particle size can be reduced such that at least 80%, 85%, 90%, or 95% of the particles have a particle size from about 5 microns to about 800 microns, from about 100 microns to about 800 microns, from about 5 microns to about 500 microns, or from about 100 microns to about 500 microns. In some embodiments, at least 80%, 85%, 90%, or 95% of the particles have a particle size between about 0.10 and 5.0 mm, 0.10 and 4.0 mm, 0.10 and 2.0 mm, or 0.25 to 0.85 mm.

In some embodiments, the conditions sufficient to dissolve water-soluble sugars comprise a temperature of from about 25 to about 100 degrees C.

The biomass can be pretreated to render the biomass more susceptible to hydrolysis. Thus, in some embodiments, the pretreatment conditions comprise elevated temperature and pressure compared to ambient temperature and pressure. In some embodiments, the pretreatment conditions include contacting the biomass (e.g., second solids phase) with hydrolytic enzymes. In some embodiments, the hydrolytic enzymes comprise one or more of cellulase, hemicellulase or an oxidoreductase such as lignin peroxidase.

The methods also provide for recycling of the first and/or second liquid phases to other steps or stages of the process. For example, at least a portion of the first liquid phase comprising the water-soluble sugars can be recycled and mixed with the second solids phase prior to or during the saccharification step (e). In some embodiments, at least a portion of the first liquid phase comprising the water-soluble sugars is contacted with additional biomass, and the additional biomass is processed as described herein.

In some embodiments, the relative amount of the first liquid phase contacting the additional biomass is about 20% to 90%, 30% to 80%, 40% to 70%, 50% to 60%, 60% to 70%, 70% to 80% or about 50% to 80% by weight

In some embodiments, at least a portion of the first liquid phase comprising the water-soluble sugars is mixed with the fermentable sugars produced during the saccharification stage. The fermentable sugars can be fermented to produce ethanol or other valuable products.

The second liquid phase can also be recycled. In one embodiment, at least a portion of the second liquid phase is contacted with an additional solids phase prior to or during the pretreatment step (c). In some embodiments, at least a portion of the first liquid phase, at least a portion of the second liquid phase, or at least a portion of the first liquid phase and at least a portion of the second liquid phase is recycled by contacting the biomass with the at least a portion of the first liquid phase, the at least a portion of the second liquid phase, or the at least a portion of the first liquid phase and the at least a portion of the second liquid phase.

In some embodiments, the particle size of the biomass is reduced prior to contacting the biomass with the first aqueous liquid in step (a). For example, in some embodiments, the particle size is reduced such that at least 80%, 85%, 90%, or 95% of the particles have a particle size between about 0.1 and 5.0 mm, 0.1 and 4.0 mm, or 0.1 and 2.0 mm.

In some embodiments, the solids concentration of the first solids phase after mixing with the second aqueous liquid is between about 1% and 50%, 5% and 45%, 10% and 30%, or 15% and 25% by weight.

In some embodiments, the solids concentration of the biomass (e.g., second solids phase) that is subjected to conditions sufficient to produce fermentable sugars (saccharification) is at least 10%, at least 15%, at least 20%, at least 25%, at least 30%, at least 35%, at least 40%, at least 45%, or at least 50% by weight.

In some embodiments, the method further comprises subjecting the first solids phase to one or more additional washing steps, wherein the wash liquid is recycled and added to the biomass during the initial wash step (a) or the pretreatment step (c). In one embodiment, the second solids phase is subjected to one or more additional washing steps, wherein the wash liquid is recycled and added to the biomass during the pretreatment step (c).

The biomass can be processed to remove sand and other solid contaminants prior to separating the first aqueous liquid from the biomass. The first liquid phase comprising the water soluble sugars can also be processed to remove sand and other solid contaminants.

In some embodiments, the washed biomass from the initial wash step (a) is adjusted to a pH of between 3 and 6.5. In some embodiments, the biomass from the pretreatment step (c) is adjusted to a pH of between 3 and 6.5.

After pretreatment, the liquid phase contains dissolved inhibitors of fermentation. Thus, in another embodiment, at least a portion of the second liquid phase is further concentrated and burned to recover energy from one or more dissolved inhibitors of fermentation. In some embodiments, at least a portion of the second liquid phase is concentrated and/or purified to recover one or more dissolved inhibitors of fermentation.

In some embodiments, at least a portion of the first liquid phase comprising the water-soluble sugars is concentrated and fermented. In some embodiments, the fermentable sugars produced by the saccharification step are separated from residual solids to produce a sugar syrup. The residual solids can also be washed one or more times to recover additional sugars. In some embodiments, the residual solids are burned. In one embodiment, the fermentable sugars are separated from residual solids prior to fermentation.

In some embodiments, the method is a continuous process. In some embodiments, the method is a batch process.

The disclosure also provides a processed biomass produced by the methods described herein.

### DEFINITIONS

Unless defined otherwise, all technical and scientific terms used herein have the same meaning as commonly understood by one of ordinary skill in the art to which this disclosure pertains. Although essentially any methods and materials similar to those described herein can be used in the practice or testing of the present methods, only exemplary methods and materials are described. For purposes of the present disclosure, the following terms are defined below.

The terms “a,” “an,” and “the” include plural referents, unless the context clearly indicates otherwise.

The term “biomass” or “biomass feedstock” refers to any material comprising lignocellulosic material. Lignocellulosic materials are composed of three main components: cellulose, hemicellulose, and lignin. Cellulose and hemicellulose contain carbohydrates including polysaccharides and oligosaccharides, and can be combined with additional components, such as protein and/or lipid. Examples of biomass include agricultural products such as grains, e.g., corn, wheat and barley; sugarcane; corn stover, corn cobs and other inedible waste parts of food plants; grasses such as switchgrass; and forestry biomass, such as wood and waste wood products.

The term “lignocellulosic” refers to material comprising both lignin and cellulose, and may also contain hemicellulose.

The term “cellulosic,” in reference to a material or composition, refers to a material comprising cellulose.

The term “saccharification” refers to production of fermentable sugars from polysaccharides by hydrolytic enzymes. Examples of hydrolytic enzymes include cellulase and hemicellulase. Hydrolytic enzymes are also referred to as “saccharification enzymes.”

The term “fermentable sugar” refers to a sugar that can be converted to ethanol or other valuable products during fermentation, for example during fermentation by yeast. For example, glucose is a fermentable sugar derived from hydrolysis of cellulose, whereas xylose, arabinose, mannose and galactose are fermentable sugars derived from hydrolysis of hemicellulose.

The term “simultaneous saccharification and fermentation (SSF) refers to providing saccharification enzymes during the fermentation process. This is in contrast to separate hydrolysis and fermentation (SHF) steps.

The term “pretreatment” refers to treating the biomass with physical, chemical or biological means, or any combination thereof, to render the biomass more susceptible to hydrolysis, for example, by saccharification enzymes. Pretreatment can comprise treating the biomass at high pressure and/or high temperature. Pretreatment can further comprise physically mixing and/or milling the biomass in order to reduce the size of the biomass particles. Devices that are useful for physical pretreatment of biomass include, e.g., a hammermill, shear mill, cavitation mill or colloid or other high sheer mill. An exemplary colloid mill is the Cellunator™ (Edeniq, Visalia, Calif.). Reduction of particle size is described in, for example, WO2010/025171.

The term “pretreated biomass” refers to biomass that has been subjected to pretreatment to render the biomass more susceptible to hydrolysis.

The term “elevated pressure,” in the context of a high pressure and high temperature (HPHT) pretreatment step, refers to a pressure above atmospheric pressure (e.g., 1 atm at sea level) based on the elevation, for example at least 20, 30, 40, 50, 60, 70, 80, 90, 100, 110, 120, 130, 140, or 150 psi or greater at sea level.

The term “elevated temperature,” in the context of a high pressure and high temperature (HPHT) pretreatment step, refers to a temperature above ambient temperature, for example at least 100, 110, 120, 130, 140, 150, 160, 170, 180, 190, or 200 degrees C. or greater. When used in HPHT pretreatment, the term includes temperatures sufficient to substantially increase the pressure in a closed system. For example, the temperature in a closed system can be increased such that the pressure is at least 100 psi or greater, such as 110, 120, 130, 140, 150 psi or greater.

The term “hydrolysis” refers to breaking the glycosidic bonds in polysaccharides to yield simple monomeric and/or oligomeric sugars. For example, hydrolysis of cellulose produces the six carbon (C6) sugar glucose, whereas hydrolysis of hemicellulose produces the five carbon (C5) sugars xylose and arabinose. Hydrolysis can be accomplished by acid treatment or by enzymes such as cellulose, β-glucosidase, and xylanase.

The term “inhibitor” refers to a compound that inhibits the fermentation process. Inhibitors can be sugar degradation products that result from pretreatment of lignocellulose and/or cellulose. Examples of inhibitors include 2-furoic acid, 5-HMF, furfural, 4-HBA, syringic acid, vanillin, syringaldehyde, p-coumaric acid, and ferulic acid.

### BRIEF DESCRIPTION OF THE DRAWINGS

FIG. 1 shows a general schematic of one illustrative embodiment of the method. In this schematic, biomass enters the process at the top left [A], where it is preprocessed by grinding to the desired particle size. The ground biomass is then sent to a hot wash and mixed with fresh water [B] and process recycle [C]. After a residence time to be determined by process factors such as composition and particle size, the slurry is sent to a screw press [K] and the pressate liquid [C] is recycled with a small purge stream [E] comprising the recovered sugars sent forward to saccharification or fermentation. The press cake [D] is sent to pretreatment, where it is combined with pretreatment recycle water [G] and fresh water [J] as needed. Following pretreatment, the slurry [F] is pumped through a colloid mill [L] using pump [M] and into another screw press [K]. The pressate liquid [G] is recycled, with a purge stream [I] sent separately to (in this example) combustion to recover energy from the dissolved organics. The cake [H] is mixed with sugar stream [E] and sent to saccharification.

FIG. 2 shows a flowchart of one illustrative embodiment of the method. The values of solids, sugars, and inhibitors shown are based on a model using the indicated input feedstock values.

FIG. 3 shows a flowchart of another illustrative embodiment of the method showing additional wash steps. The values of solids, sugars, and inhibitors shown are based on a model using the indicated input feedstock values.

FIG. 4 shows the experimental flow and mass balance of the solids and total mass for each step during the process. Additionally, the total solids (TS) for each stream is reported as determined using microwave solids analysis.

FIG. 5 shows the sugar/EtOH profile during fermentation of pressed liquor and press liquor that had been concentrated to 4× using a rotovap. In both cases the sugars were consumed in the first 24 hours.

FIG. 6 shows the saccharification efficiency for control corn stover and washed and pressed corn stover. The Accelerase Trio enzyme packaged was used for all saccharification. The legend displays the percent trio with respect to glucan that was dosed.

FIG. 7 shows the acetic acid, furfural, and HMF formation for corn stover compared to the washed and pressed corn stover.

FIG. 8 shows a schematic for the model of the biomass wash.

FIG. 9 shows the yield of the sugar and liquid as a function of purge ratio. The yield was defined as 100% for the process where all the liquor from the screw press is purged from the system (i.e. 100% purge ratio).

FIG. 10 shows the concentration of glucose and xylose as a function of the purge ratio.

FIG. 11 shows the mass flow rate as a function of the purge ratio for a 1.0 ton/day ethanol plant.

### DETAILED DESCRIPTION OF THE INVENTION

The present application discloses methods of processing biomass that includes one or more recycled loops of liquid streams that are used to dilute the biomass at various stages of the process. The methods described herein surprisingly result in increased saccharification efficiency and production of sugars from biomass, with the potential to increase revenue for an ethanol production facility. The process involves the initial step of soaking or washing the biomass with an aqueous liquid, such as water, to dissolve water-soluble sugars and sugar precursors (e.g., polysaccharides) that are associated with the biomass. Removal of the water-soluble sugars from the biomass is desirable, as sugars thermally decompose into inhibitors of the microbiological activity (e.g., yeast) that is useful for subsequent fermentation of the biomass. The slurry of aqueous liquid and at least a portion of the biomass is then separated into a first liquid phase and a first solids phase. The first liquid phase comprising the water-soluble sugars is then recycled or “looped” back to the soaking step, where it can be combined with the biomass. At least a portion of the first liquid phase comprising the water-soluble sugars can be sent to the saccharification step, where it can be used to dilute downstream solids prior to saccharification. Alternatively, at least a portion of the first liquid phase comprising the water-soluble sugars can be sent directly to the fermentation step. Additional aqueous liquid can be added to the biomass, as necessary to form a slurry of the desired consistency. The slurry is then again separated into a liquid phase and a solids phase, and the process is repeated. The process can be a batch process or a continuous process.

In subsequent steps, the solids phase can be diluted with aqueous liquid and pretreated under conditions that render the biomass more susceptible to hydrolysis. In some embodiments, the pretreatment step does not release a significant amount of sugars from the biomass. The pretreated biomass slurry can be further processed to produce a relatively uniform particle size. The pretreated biomass is then separated into a second liquid phase and a second solids phase. The second liquid phase typically contains low amounts of fermentable sugars, but does contain organic compounds that can function as inhibitors of fermentation. The second liquid phase can be recycled or looped back to the pretreatment step, where it is combined with additional biomass and, optionally, additional aqueous liquid, as necessary to dilute the biomass and form a slurry of the desired consistency. The biomass slurry containing the recycled second liquid phase is then pretreated as above, and the process is repeated. The second liquid phase containing inhibitors of fermentation is typically not used to wash or soak the biomass at the initial wash step described above, as this would introduce inhibitors back into the first liquid phase, defeating the purpose of sending at least a portion of this first liquid phase directly to fermentation.

The first and/or second liquid phase streams can be further separated into a wash stream that is used to dilute biomass at various stages of the process, and a purge stream. Purge streams from the two liquid recycle loops can be treated as desired. For example, the purge stream from the first liquid phase containing water soluble sugars from the initial wash step can be sent to fermentation. The purge stream from the second liquid phase containing organic inhibitors after pretreatment of the biomass can be concentrated and burned, or the compounds can be recovered. In general, the second liquid phase containing inhibitors is not used to dilute solids prior to saccharification, and is not added to a fermenter.

The second solids phase can then be subjected to conditions sufficient to produce fermentable sugars. For example, the second solids phase can be treated with hydrolytic enzymes to release fermentable sugars from polysaccharides in the biomass (e.g., saccharification). In some embodiments, the concentration of solids that is subject to saccharification using the methods described herein is substantially greater than that obtainable in prior methods for processing biomass. In some embodiments, the concentration of solids that is subject to saccharification is about three-fold greater than that in prior processes.

The methods described herein have the following advantages over prior methods for processing biomass to produce ethanol or other valuable products. First, the separation of free sugars from the biomass prior to pretreatment at high temperatures can reduce the amount of at least some of the inhibitors formed during the pretreatment step. Second, the dilute solids stream results in a lower concentration of solids in the slurry, which results in less wear and tear on moving parts in the ethanol production facility. Third, the method allows for higher quantities of sugars to be recovered and sent to fermentation, as the sugars could otherwise be destroyed during pretreatment. Fourth, the method allows for higher concentrations of biomass solids to be treated during saccharification and/or fermentation of the biomass, which increases the ethanol yield. Although dilute preprocessing requires larger scale equipment than more concentrated processing methods, both the soak and pretreatment steps require low residence times compared to saccharification and fermentation—as much as 99% less. The marginally higher capital costs for larger equipment for the soak and pretreatment steps are offset by significantly reduced equipment and pump erosion and subsequent maintenance costs. In addition, downstream equipment sizes can be reduced by up to 60% versus without 2-loop processing, resulting in additional capital cost savings.

### Preprocessing of the Biomass

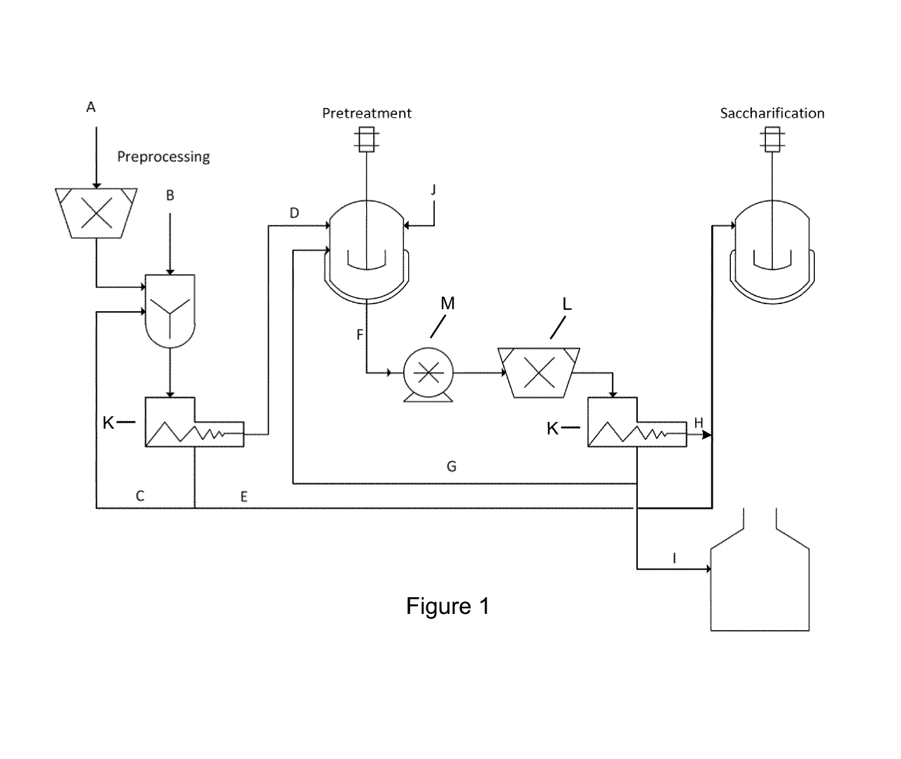

The biomass can be preprocessed by grinding the biomass to a desired particle size (see FIG. 1, step A). For example, the biomass can be ground to a particle size in the range of about 0.1 mm to about 5.0 mm, about 0.1 mm to about 4.0 mm, or about 0.1 mm to about 2.0 mm. In some embodiments, the particle size can be selected such that the particles pass through a hammer mill during or after the pretreatment step described below.

The preprocessed (e.g., ground) biomass can also be mixed with water to form a slurry of the desired consistency. For example, the slurry can be in the range of about 20% solids to about 100% solids by weight, e.g., about 20%, 30%, 40%, 50%, 60%, 70%, 80%, 90% or 100% solids by weight. In some embodiments, the ground biomass is not mixed with water or other aqueous liquid, and is substantially dry. The preprocessed biomass is then introduced into the first loop of the process.

### The First Loop

As shown in FIG. 1, in the first loop of the method, the biomass is washed or soaked with an aqueous liquid at atmospheric pressure to dissolve free sugars and sugar precursors (e.g., xylan) that are associated with the biomass (see step B of FIG. 1). The step of washing the biomass is sometimes referred to as “wet preprocessing.” In some embodiments, the aqueous liquid is water. In some embodiments, acid is added to the aqueous liquid to decrease the pH. In some embodiments, the addition of the aqueous liquid to the biomass forms a dilute biomass slurry. In some embodiments, the dilute biomass slurry can have a solids content of about 1% to about 30% solids by weight, for example about 5%, 10%, 15%, 20%, 25% and 30% solids by weight. The temperature of the wash can be between about 25 and 100 degrees C., for example, about 30° C., 40° C., 50° C., 60° C., 70° C., 80° C., 90° C., or 100° C. It will be understood that the temperature of the wash can vary with the duration of the wash or soak step, such that a higher temperature results in a shorter time necessary for the sugars to dissolve in the aqueous liquid. In some embodiments, the duration of the wash step is from about 10 to about 60 minutes. In some embodiments, the duration of the wash step can be up to about 4 hours.

In some embodiments, the temperature of the wash or soak is less than the temperature that results in thermal degradation of sugars into inhibitors of fermentation. Thus, in some embodiments, the temperature of the initial wash is less than about 150 degrees C.

The washed biomass is then treated to physically separate the aqueous liquid comprising the dissolved sugars from the biomass solids. In some embodiments, the washed biomass is passed through a screw press which separates the biomass slurry into a pressate liquid phase and a press cake solids phase (see FIG. 1, “C” and “D”). However, any suitable method for separating the biomass into a liquid phase and a solids phase can be used, including, without limitation, centrifugation, settlement, decanting, screens, filtration, and flocculation. In some embodiments, the liquid phase is recycled to dilute the biomass during other steps in the process. For example, in one embodiment, the liquid phase or a portion thereof is used to dilute the biomass in the initial “wet preprocessing” wash step. Thus, in one embodiment, the biomass is contacted with the liquids phase at a temperature sufficient to dissolve additional free sugars associated with the biomass. The biomass that is washed with the recycled liquid phase can be further mixed with fresh aqueous liquid (e.g. water) as necessary to form a slurry of the desired consistency and solids content. Thus, in some embodiments, the aqueous liquid comprises fresh water, the liquid phase, or a combination of both. The aqueous liquid can also comprise liquids from other downstream steps in the process.

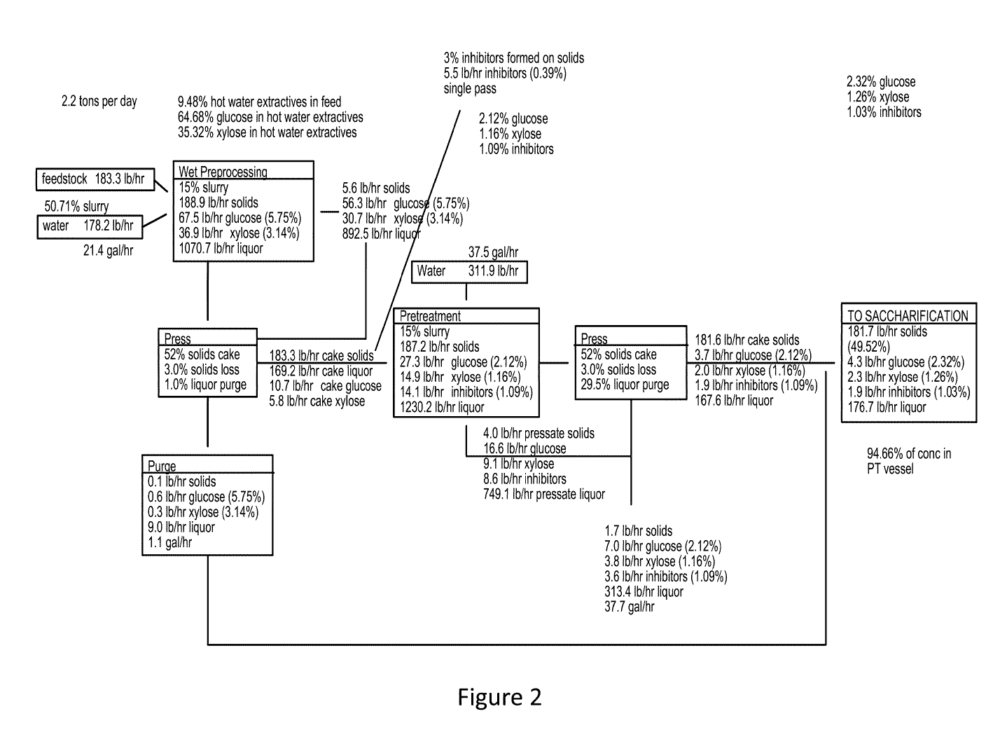

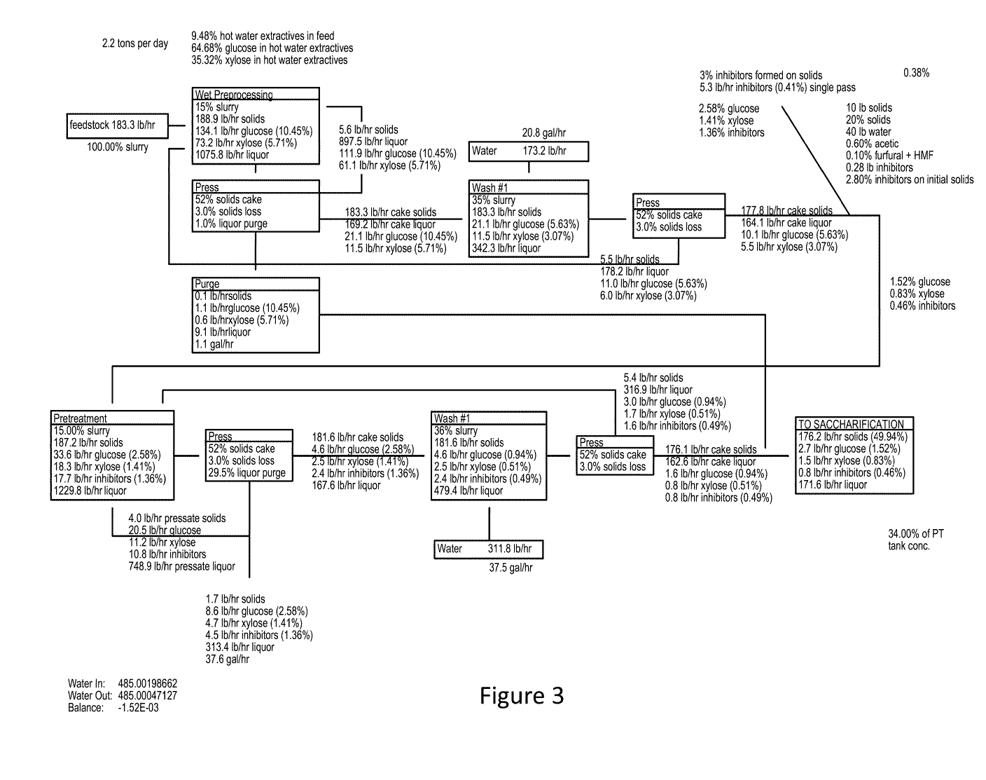

The liquid phase can also be combined with biomass in subsequent, downstream process steps as well. For example, as shown in FIGS. 1-3, in some embodiments, a portion of the liquid phase containing dissolved sugars is separated into a purge stream (see FIG. 1, “E”), and the purge stream “sent forward” where it is added to the biomass during the saccharification and/or fermentation step. This provides additional sugars that can be converted to ethanol. In some embodiments, the liquid phase and/or purge stream contains about 0.5% to about 10% glucose (e.g., about 1%, 2%, 3%, 4%, 5%, 6%, 7%, 8%, 9% or 10% glucose) by weight, and about 0.5% to about 10% xylose (e.g., about 0.5%, 1%, 2%, 3%, 4%, 5%, 6%, 7%, 8%, 9% or 10% xylose) by weight. Representative examples of the amounts sugars in the liquid phase and purge phase are shown in FIGS. 2 and 3. The liquid phase and/or purge stream contains low amounts of inhibitors of fermentation. The purge stream can also be used to dilute the biomass as necessary to allow for efficient enzyme mixing during saccharification, or the purge stream can be added directly to the fermenters. The liquid phase or a portion thereof (e.g., the purge stream) can be concentrated to minimize the amount of water added to the saccharification and/or fermentation steps. The liquid phase or portion thereof can be concentrated by methods know in the art, including but not limited to evaporation and membrane filtration.

In some embodiments, the biomass is processed in a batch process. For example, the biomass that is washed to remove free sugars can be processed as a single batch during the first loop, and the liquids phase from the first biomass batch used to wash a second, distinct biomass that is preprocessed and washed as a second biomass batch.

In some embodiments, the biomass is processed in a continuous process. For example, a continuous stream of biomass feedstock can be washed to remove free sugars and produce a continuous stream of biomass slurry that is sent to the first separation step. The separation step can produce a continuous stream of the first liquid phase that is recycled to wash the biomass feedstock, and a continuous first solids phase stream that is sent to pretreatment.

In some embodiments, the biomass is processed to remove sand and other solid contaminants prior to or during separating the first aqueous liquid from the biomass. In one embodiment, the biomass that is processed to remove sand and other solid contaminants has a solids content of about 1% to about 5% solids. In some embodiments, the first liquid phase is processed to remove sand and other solid contaminants. The sand and other solid contaminants can be removed by mechanical separation, including but not limited to, filters, screens, settlement and hydrocyclones.

In some embodiments, the biomass slurry is chemically modified. For example, in some embodiments, the biomass slurry can be chemically modified to adjust the pH. In some embodiments, the pH of the biomass is adjusted to a pH of between about 3.0 and about 6.0. In some embodiments, the pH of the slurry generated by contacting the preprocessed biomass with an aqueous liquid is adjusted to a pH of between about 3.0 and about 6.5.

In some embodiments, the solids phase (e.g., the press cake) is then pretreated to render the biomass solids more susceptible to hydrolysis, as described below. In some embodiments, the press cake is about 30% to about 60% solids by weight, e.g., about 30%, 35%, 40%, 45%, 50%, 55% or 60% solids by weight. In one embodiment, the press cake is about 52% solids by weight. FIG. 2 shows expected values for solids, sugars and inhibitors at various stages of the process based on one embodiment using the indicated amount of input feedstock.

### I. Optional Wash Step

As shown in FIG. 3, the solids phase (“cake solids”) can be subjected to one or more additional wash steps prior to pretreatment, and the wash liquid recycled to various steps in the process. The additional wash steps increase the efficiency of sugar recovery from the biomass. In the additional wash steps, the solids phase is mixed with water to form a slurry (e.g., of about 15-50% solids by weight), and the slurry subjected to another separation step to produce a liquid phase and a solids phase. In some embodiments, the concentration of solids in the slurry is about 15%, 20%, 25%, 30%, 35%, 40%, 45% or 50% solids by weight. In one embodiment, the separation step is accomplished by using a screw press to produce a pressate liquid phase and a press cake solids phase. In one embodiment, the press cake is about 30%, 35%, 40%, 45%, 50%, 55%, 60%, or about 65% solids by weight after the optional wash and separation steps. In one embodiment, the press cake is at least about 50% solids by weight after the optional wash and separation steps. This optional wash results in the recovery of more sugars from the solids phase prior to pretreatment, and has the added benefit of decreasing the amount of sugars which may be converted into inhibitors produced by thermal degradation of sugars during pretreatment. The liquid phase can then be recycled to upstream steps of the process. For example, the liquid pressate can be used as “makeup water” and mixed with the biomass at the wet preprocessing step described herein. Thus, in some embodiments, the biomass is mixed with the liquid pressate from the first separation step and with the liquid pressate from the second separation step. This allows for the biomass feedstock from the preprocessing step to be diluted with the recycled liquid phases, thereby reducing the amount of fresh water required to dilute the biomass feedstock in order to form a slurry of the desired consistency. The recycled liquid phase can also be used to dilute the biomass at any other stage of the process, as desired. The additional wash step(s) can also increase the sugar recovery efficiency by improving the dissolved/suspended solids separation (e.g., each round of pressing the solids to remove wash liquid that contains dissolved sugars increases the sugar recovery efficiency).

### Pretreatment

As shown in FIGS. 1-3, the biomass can be pretreated to render the lignocellulose and cellulose more susceptible to hydrolysis. In some embodiments, pretreatment comprises subjecting the biomass to elevated temperatures and pressure in order to render the lignocellulose and cellulose accessible to enzymatic hydrolysis. In some embodiments, the temperature and pressure are increased to amounts and for a time sufficient to render the cellulose susceptible to hydrolysis. In some embodiments, the pretreatment conditions can comprise a temperature in the range of about 150° C. to about 210° C. The pretreatment temperature can be varied based on the duration of the pretreatment step. For example, for a pretreatment duration of about 60 minutes, the temperature is about 160 degrees C.; for a duration of 30 minutes, the temperature is about 170 degrees C.; for a duration of 5 minutes, the temperature is about 210 degrees C.

The pretreatment conditions can also comprise increased pressure. For example, in some embodiments, the pressure can be at least 100 psi or greater, such as 110, 120, 130, 140, 150 psi or greater. In some embodiments, the biomass is pretreated in a closed system, and the temperature is increased in an amount sufficient to provide the desired pressure. In one embodiment, the temperature is increased in the closed system until the pressure is increased to about 125 to about 145 psi. Persons of skill in the art will understand that the temperature increase necessary to increase the pressure to the desired level will depend on various factors, such as the size and shape of the closed system. In some embodiments, pretreatment comprises any other method known in the art that renders lignocellulose and cellulose more susceptible to hydrolysis, for example, acid treatment, alkali treatment, and steam treatment, or combinations thereof.

In some embodiments, the pretreatment step does not result in the production of a substantial amount of sugars. For example, in some embodiments, pretreatment results in the production of less than about 10%, 5%, 1%, 0.1%, 0.01%, or 0.001% by weight glucose, less than about 10%, 5%, 1%, 0.1%, 0.01%, or 0.001% by weight xylose, and/or less than about 10%, 5%, 1%, 0.1%, 0.01%, or 0.001% by weight sugars in general. In some embodiments, the amount of sugars in the process stream entering the pretreatment stage is substantially the same as the amount of sugars in the process stream exiting the pretreatment stage. For example, in some embodiments, the difference between the amount of sugars in the process stream entering the pretreatment stage and the amount of sugars exiting the pretreatment stage is less than about 10%, 5%, 1%, 0.1%, 0.01%, or 0.001% by weight.

In some embodiments, the biomass that is pretreated comprises the solids phase produced as described above in the first loop. Thus, in one embodiment, the solids phase is contacted with a second aqueous liquid under conditions sufficient to render the solids more susceptible to hydrolysis. In some embodiments, the second aqueous liquid is fresh water. In some embodiments, the second aqueous liquid is a recycled liquid, as described herein. The addition of the liquid to the biomass can form a dilute slurry (see FIG. 1, “F”). For example, the slurry can comprise between about 1% and 50%, 5% and 45%, 10% and 30%, or 15% and 25% solids by weight. In one embodiment, the slurry is about 10%, 11%, 12%, 13%, 14%, 15%, 16%, 17%, 18%, 19%, 20%, 21%, 22%, 23%, 24%, or 25% solids by weight. The amount of solids can be adjusted as desired both before and after the pretreatment step.

In other embodiments, the biomass that is pretreated comprises a mixture of biomass solids that are treated as part of a continuous process. Thus, the pretreatment step can contain a mixture of biomass solids that are produced sequentially or in parallel. For example, in some embodiments, the pretreatments step comprises one or more press cakes from the first loop or one or more press cakes from the optional wash step described above.

As shown in FIG. 1, in some embodiments, pretreatment can further comprise physically mixing and/or milling the biomass in order to reduce the size of the biomass particles. The yield of biofuel (e.g., ethanol) can be improved by using biomass particles having relatively small sizes. Devices that are useful for physical pretreatment of biomass include, e.g., a hammermill, shear mill, cavitation mill or colloid or other high sheer mill. Thus, in some embodiments, the pretreatment step comprises physically treated with a colloid mill. An exemplary colloid mill is the Cellunator™ (Edeniq, Visalia, Calif.). In some embodiments, the biomass is physically pretreated to produce particles having a relatively uniform particle size of less than about 1600 microns. For example, at least about 50%, 60%, 70%, 80%, 85%, 90%, or 95% of the pretreated biomass particles can have a particle size from about 100 microns to about 800 microns. In some embodiments, at least about 50%, 60%, 70%, 80%, 85%, 90%, or 95% of the pretreated biomass particles have a particle size from about 100 microns to about 500 microns. In some embodiments, the pretreatment step does not involve the use of acids which can degrade sugars into inhibitors of fermentation.

In some embodiments, the pH of the pretreated biomass is adjusted to a pH of between about 3.0 and about 6.5. In some embodiments, the pH of the biomass is adjusted during or after the pretreatment step to be within the optimal range for activity of saccharification enzymes, e.g., within the range of about 4.0 to 6.0. In some embodiments, the pH of the biomass is adjusted using Mg(OH)₂, NH₄OH, or a combination of Mg(OH)₂ and NH₄OH.

### The Second Loop

Referring to FIG. 1, after the pretreatment step, the biomass slurry is separated into a second liquid phase and a second solids phase. In some embodiments, a screw press is used to separate the biomass slurry into a second pressate liquid phase (“G”) and a second press cake solids phase (“H”). However, any method for separating the solids phase from the liquids phase can used, as described herein. The second liquids phase (G) can be recycled to dilute the biomass during various steps of the pretreatment process. For example, the second liquid phase can be mixed with the first solids phase prior to, during or after the pretreatment step.

In some embodiments, at least a portion of the second liquid phase can be separated into a second purge stream (see FIG. 1, “I”). The second purge stream can be concentrated and combusted to recover energy from the dissolved organic compounds. In one embodiment, the dissolved inhibitors and other compounds in the second liquid phase and/or second purge stream can be recovered and/or purified. If desired, the recovered compounds can be sold to provide an additional revenue source for the ethanol plant.

The second loop can also be performed as a batch or continuous process. For example, in a batch process, the biomass can be pretreated as a single batch (e.g., a single press cake), and the second liquid phase from the second loop is recycled and combined with another, distinct biomass (e.g., a second press cake) that is then pretreated. In one embodiment of a continuous process, the second liquid phase is recycled and combined with a mixture of biomass that is continuously added to the pretreatment vessel (e.g., from multiple press cakes). The second liquid phase can also be continuously produced from the pretreated biomass, for example, from a screw press that operates continuously, and continuously recycled to dilute the biomass during the pretreatment steps. However, because the second liquid phase from the second loop contains inhibitors of fermentation, it is generally not recycled to dilute biomass in the first loop (i.e., the “wet preprocessing” step) or sent to saccharification and/or fermentation.

In some embodiments, the second liquid phase contains reduced inhibitors of fermentation as compared to the amount of inhibitors in the first solids phase after pretreatment. For example, in one embodiment, the second liquids phase contains at least about 10%, 20%, 30%, 40%, 50%, 60%, 70%, 80%, or 85% less total inhibitors than the first solids phase after pretreatment. In some embodiments, the second liquids phase contains reduced amounts of inhibitors compared to biomass that is not separated into a second liquids phase and a second solids phase after pretreatment. The second liquids phase can be diluted (e.g., with the first loop liquid phase “liquor” or fresh water) to reduce the concentration of inhibitors in the second loop liquid stream as desired.

In some embodiments, the second liquid phase contains reduced amounts of sugars as compared to the amount of sugars in the first liquid phase of the first loop. For example, in some embodiments, the second liquid phase contains less than about 5%, less than about 4%, less than about 3%, and less than about 2% glucose by weight. In some embodiments, the second liquid phase contains less than about 5%, less than about 4%, less than about 3%, less than about 2%, and less than about 1% xylose by weight.

### II. Second Optional Wash Step

As described above for the first solids phase, the second solids phase can be subjected to one or more additional wash steps after pretreatment, and the wash liquid recycled to the pretreatment step (see FIG. 3). For example, the second press cake can be mixed with water to form a slurry (e.g., of about 30-40% solids by weight), and the slurry subjected to another separation step to produce a liquid phase and a solids phase. In one embodiment, the separation step is accomplished by using a screw press to produce a pressate liquid phase and a press cake solids phase. In one embodiment, the solids phase is about 30% to about 60% solids by weight after the additional wash and separation steps. In one embodiment, the solids phase is at least about 50% solids by weight after the additional wash and separation steps.

The liquid phase from the additional wash and separation step(s) can be recycled to dilute the biomass during the pretreatment stage. However, because the liquid phase from the second additional wash step contains inhibitors of fermentation, it is generally not recycled to the first loop or sent to saccharification and/or fermentation.

### Saccharification and Fermentation Step

After the second loop processing steps, the second solids phase can be subjected to saccharification and fermentation under conditions sufficient to produce ethanol from the biomass. In some embodiments, the saccharification conditions include contacting the solids phase biomass with hydrolytic enzymes including cellulase and hemicellulase in order to produce fermentable sugars from polysaccharides in the biomass. The fermentation conditions include contacting the solids phase biomass with yeast that are capable of producing ethanol from sugars. In some embodiments, the biomass is about 30% to about 65% solids, about 35% to about 55% solids, or about 40% to about 50% solids by weight when subjected to saccharification and/or fermentation. In some embodiments, the biomass is about 20%, 25%, 30%, 35%, 40%, 45%, 50%, 55%, 60%, or about 65% solids by weight when subjected to saccharification and/or fermentation. In one embodiment, the biomass is at least about 50% solids by weight when subjected to saccharification and/or fermentation.

The second solids phase can be combined with all or a portion of the first liquid phase prior to, during, or after the saccharification and/or fermentation steps. For example, the first liquid phase can be used to dilute the second solids phase prior to saccharification in order to aid enzyme mixing. Alternatively, the first liquid phase can be added directly to the fermenters under conditions sufficient to produce ethanol from the fermentable sugars. In some embodiments, the first liquid phase is concentrated to reduce the amount of water prior to fermentation.

Examples of enzymes that are useful in saccharification of lignocellulosic biomass include glycosidases, cellulases, hemicellulases, starch-hydrolyzing glycosidases, xylanases, ligninases, and feruloyl esterases, and combinations thereof. Glycosidases hydrolyze the ether linkages of di-, oligo-, and polysaccharides. The term cellulase is a generic term for a group of glycosidase enzymes which hydrolyze cellulose to glucose, cellobiose, and other cello-oligosaccharides. Cellulase can include a mixture comprising exo-cellobiohydrolases (CBH), endoglucanases (EG) and β-glucosidases (βG). Specific examples of saccharification enzymes include carboxymethyl cellulase, xylanase, β-glucosidase, β-xylosidase, and α-L-arabinofuranosidase, and amylases. Saccharification enzymes are commercially available, for example, Pathway™ (Edeniq, Visalia, Calif.), Cellic® CTec2 and HTec2 (Novozymes, Denmark), Spezyme® CP cellulase, Multifect® xylanase, and Trio® (Genencor International, Rochester, N.Y.). Saccharification enzymes can also be expressed by host organisms, including recombinant microorganisms.

The saccharification reaction can be performed at or near the temperature and pH optimum for the saccharification enzymes used. In some embodiments of the present methods, the temperature optimum for saccharification ranges from about 15 to about 100° C. In other embodiments, the temperature range is about 20 to 80° C., about 35 to 65° C., about 40 to 60° C., about 45 to 55° C., or about 45 to 50° C. The pH optimum for the saccharification enzymes can range from about 2.0 to 11.0, about 4.0 to 6.0, about 4.0 to 5.5, about 4.5 to 5.5, or about 5.0 to 5.5, depending on the enzyme.

The enzyme saccharification reaction can be performed for a period of time from about several minutes to about 250 hours, or any amount of time between. For example, the saccharification reaction time can be about 5 minutes, 10 minutes, 30 minutes, 60 minutes, or 2, 4, 6, 8, 12, 16, 18, 24, 36, 48, 60, 72, 84, 96, 108, 120, 130, 140, 150, 160, 170, 180, 190, 200, 210, 220, 230, 240 or 250 hours. In other embodiments, the saccharification reaction is performed with mixing or shaking to improve access of the enzymes to the cellulose. The mixing or shaking can be, for example, at 50 to 250 rpm. The specific saccharification conditions will be evident to one skilled in the art depending upon scale and other factors.

The amount of saccharification enzymes added to the reaction can be adjusted based on the cellulose content of the biomass and/or the amount of solids present in a composition comprising the biomass, and also on the desired rate of cellulose conversion. For example, in some embodiments, the amount of enzymes added is based on percent by weight of cellulose present in the biomass, as specified by the enzyme provider(s). The percent of enzyme added by weight of cellulose in such embodiments can range from about 0.1% to about 20% on this basis.

In some embodiments, the biomass is combined with all or a portion of the first liquid phase (e.g., the first purge stream) and/or all or a portion of the second liquid phase (e.g., the second purge stream) to produce a composition comprising liquid and solids. In some embodiments, the biomass is combined with water to produce a composition comprising water and solids. The amount of solids in the biomass composition can be about 5% to about 60%, about 10% to about 50%, about 15% to about 40%, about 15% to about 35%, or about 17% to about 25%. In some embodiments, the amount of solids in the biomass composition is at least 5, 10, 15, 16, 17, 18, 19, 20, 21, 22, 23, 24, 25, 30, 35, 40, 45, or 50% or more solids by weight. The amount of solids can be adjusted as desired both before and after the first loop, the pretreatment step, the second loop, or saccharification and fermentation. The amount of saccharification enzymes added can be adjusted based on the solid content of the biomass composition.

In some embodiments, the fermentable sugars produced by saccharification are separated from residual solids prior to fermentation. In some embodiments, the fermentable sugars produced by saccharification are not fermented, but are instead separated from the residual solids to produce a clean sugar syrup. In some embodiments, the residual solids are subjected to one or more wash steps to recover additional sugars, and the recovered sugars are added to the clear sugar syrup. The residual solids can also be burned to generate energy for other processes in the ethanol production facility.

### EXAMPLES

### Example 1

This example shows that washing the biomass prior to pretreatment increased the saccharification yields for both glucose and xylose.

Materials and Methods

Overview of Experiments

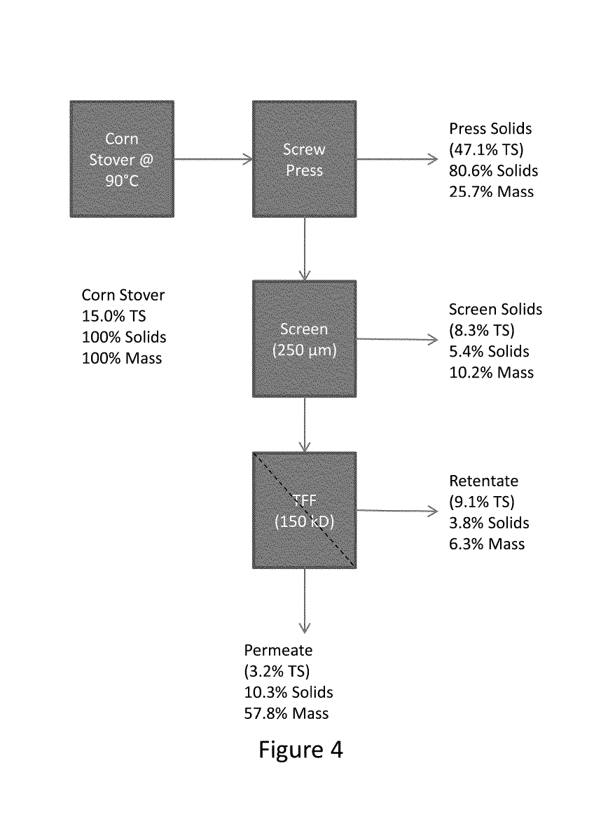

The overview of the experimental flow is shown in FIG. 4. The experiments involved soaking the corn stover at 15% total solids at 90° C. for 15 minutes. Then the biomass was pressed using a screw press (Vincent Corp, Tampa Bay, Fla.). The liquor from the screw press was passed through a 250 μm sieve (SWECO, Inc., Florence Ky.). The liquor was passed through a 150 kDa PES TFF filter (SmartFlow Technologies, Apex, N.C.) to remove any non-dissolved solids. The solids were pretreated in the 1 L bomb reactor. Saccharification was performed using the Accelerase Trio (Genecor, Rochester, N.Y.) enzyme cocktail.

Screw Press/Screen

A corn stover slurry was created at 15% total solids using the Golden State Feed corn stover. The slurry was heated to 90° C. for 15 minutes while being stirred in a jacketed tank. The slurry was pressed through a Vincent CP-4 screw press. The liquor was passed through a 250 μm mesh hand sieve. Samples of the solids were collected for compositional analysis. The press solids were taken through pretreatment in the 1 L bomb reactor and saccharification.

TFF System

An OptiSep 1000 module from SmartFlow Technologies containing a PES 150 kDa membrane with a membrane area of 0.045 m² and a channel height of 1.0 mm was used for the filtration. A recirculation rate of 9.8 LPM was used, which corresponds to a shear rate of 7,000 sec⁻¹ and a channel velocity of 1.5 m/s.

Pretreatment/Saccharification

The solids were pretreated using the 1 L bomb reactor at 175° C. for 30 min. The pressed solids were diluted from approximately 45% down to 15% TS before going into pretreatment. The control corn stover was hydrated to 15% solids before pretreatment.

The solids were saccharified using the Accelerase Trio enzyme cocktail. 100 g of 15% biomass was used in the shake flask saccharifications. The control biomass was dosed with 20% Trio based upon glucan composition. The washed and pressed solids were dosed with 18.9% Trio based upon glucan composition in the first saccharification because the wash increased the relative glucan concentration by removing ash from the sample. After compositional analysis was obtained, the second saccharification was performed using a 20% Trio based upon glucan composition dosing.

### Fermentation

The pressed liquor was fermented to illustrate the quantity of ethanol that could be produced. Because the sugar concentration was relatively low, the sugar concentration was increased by using the rotovap to increase the sugar concentration to 4× the starting quantity. The sugars were fermented in triplicate, one set using 1× sugar and the other set using the 4× concentrated sugars. Allpen and Lactrol, commonly used and well known antibiotics, were both added at 10 ppm concentration. Urea was added to 400 ppm. Yeast was used to ferment the sugars. The fermentation was carried out at 34° C. and 120 rpm.

TABLE 1
Compositional analysis of the solids streams during processing.

| | Feed | | | |
|---|---|---|---|---|
| | Corn | Press | Screen | TFF |
| Test Parameters | Stover | Solids | Solids | Retentate |
| % Glucan | 35.5 | 37.6 | 37.6 | 25.0 |
| % Xylan | 17.6 | 17.8 | 18.0 | 10.6 |
| % Galactan | 0.6 | 0.7 | 0.9 | 0.8 |
| % Arabinan | 1.4 | 3.1 | 4.0 | 3.5 |
| % Mannan | 0.0 | 0.0 | 0.0 | 0.0 |
| % Total Carbohydrates | 55.1 | 59.2 | 60.4 | 40.0 |
| % Extractives | 26.6 | 11.4 | 25.7 | 29.9 |
| % Solids | N/A | 97.4 | 96.5 | 97.4 |
| % Ash @ 575° C. after acid | 8.5 | 7.0 | 5.5 | 19.2 |
| hydrolysis | | | | |
| % Ash @ 575° C. of sample | 15.1 | 12.1 | 11.0 | 23.0 |
| % Acid Insoluble Residue (AIR) | 32.7 | 31.0 | 28.5 | 44.2 |
| % Acid Insoluble Lignin (AIL) | 24.7 | 24.0 | 23.0 | 25.0 |
| % Acid Soluble Lignin (ASL) | 1.0 | 1.1 | 1.2 | 0.8 |
| % Total Lignin | 25.7 | 25.1 | 24.2 | 25.8 |
| Sum of Carbohydrates, | 89.3 | 91.3 | 90.1 | 85.0 |
| Lignin, ASH | | | | |

% Extractives includes both hot Water and Ethanol

Results

Mass Balance/Compositional Analysis

FIG. 4 provides an overview of the mass balance of the solids and total mass through each unit operation. Several facts stand out in this mass balance. The screw press was able to recover 80.6% of the starting solids. With the additional 5.4% that the screen was able to recover, a total of 86.0% of the solids were recovered in these two steps. Additionally, the permeate out of the 150 kD filter had a solids content of 3.2% TS. Therefore, roughly 21% of the solids were dissolved in hot water.

Table 1 shows the compositional analysis for the solid streams. The composition of the press solids and screen solids were very similar, which indicates that most of press solids are larger than 250 μm. The carbohydrate concentration increased in the press solids and screen solids relative to the starting corn stover (60% versus 55%). The solids out of the TFF had a much lower carbohydrate percentage (40% versus 55%) than the feed corn stover. Additionally, the TFF solids had considerably higher ash content (23%) versus both the starting corn stover (15%) and the press solids/screen solids (12% and 11%, respectively). Because the carbohydrates were enriched in the press solids and screen solids streams, combining these two streams would provide 91.1% of the starting solids while 2.7% of the glucan would be lost in the TFF retentate stream. (Note: The other 6.2% was “lost” from the system due to the mass balance not closing. Therefore, it is possible that the overall glucan recovery would be 95+%.)

Free Sugar Recovery and Fermentation

One economic driver of doing wet preprocessing followed by solids concentration is the recovery of “free” sugars that are present in the ground corn stover. The water out of the press was analyzed via liquid chromatography (HPLC) for both glucose and xylose. The experiment was performed twice, and it was found that the glucose content was 0.47% and 0.48% and the xylose content was 0.35% and 0.28%. Therefore, the average glucose content was 3.2% of the starting solids, and the average xylose was 2.1% of the starting material. These sugars are relatively pure as nearly no inhibitors are formed during the hot water wash. The observed level of acetic acid was 0.025%. Typical levels observed of HMF, furfural, and syringaldehyde were 15 mg/1, 2 mg/l, and 83 mg/l, respectively.

During the processing, 65% of the water was recovered out of the TFF system. Therefore, roughly ⅔ of the sugars would be recoverable out of the TFF. At this point, a reverse osmosis (RO) filtration system or evaporator could be used to increase the concentration. The recovery would increase if higher concentrations were obtained in the retentate, press solids, and sieve solids. Additionally, water recycle strategies would increase the concentration of the sugars to make it so less concentration would be needed to feed the sugars to fermentation.

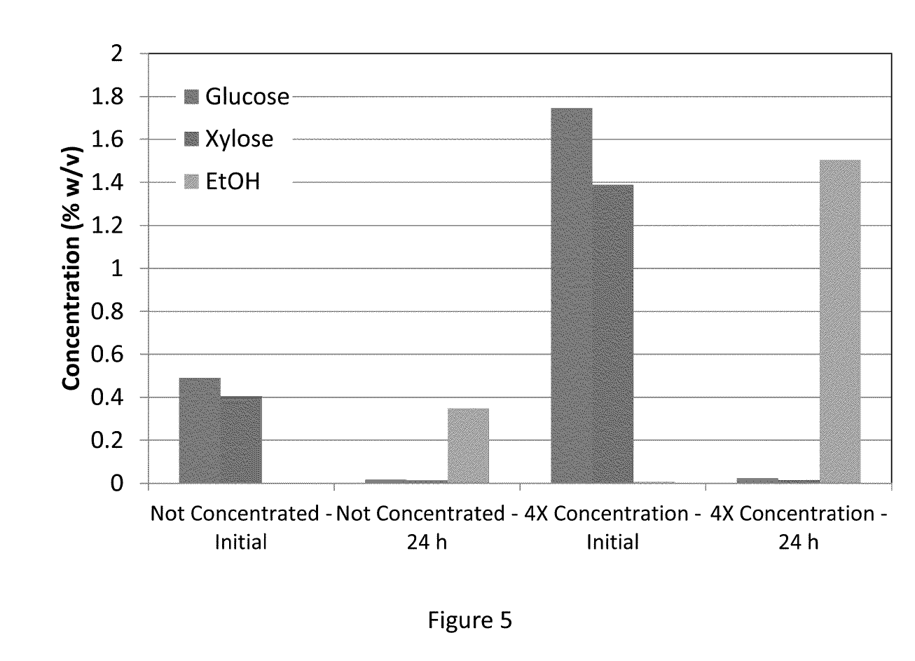

FIG. 5 illustrates the fermentation progression for both the press liquor and the 4× concentrated press liquor. Both fermentations were complete in 24 h with both the glucose and xylose consumed at this point. For the original press liquor, the EtOH conversion yield was 139% of theoretical based upon glucose and 76% based upon both glucose and xylose. For the 4× concentrated press liquor, the ethanol conversion yield was 169% of theoretical based upon glucose and 94% of theoretical based upon both glucose and xylose. The yeast strain used was not genetically engineered to ferment xylose. Therefore, the peak identified as the xylose peak in the HPLC is either a fermentable sugar (and not xylose) or the strain is capable of fermenting xylose when the inhibitor levels are low.

Saccharification of Pressed Solids

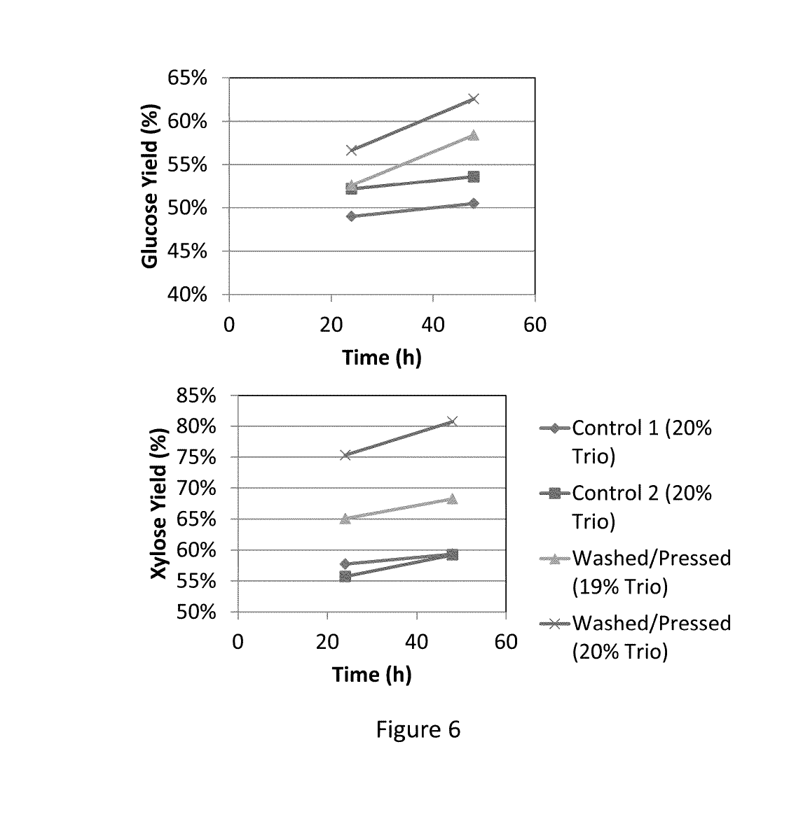

The pretreatment and saccharification of the wash/pressed biomass and a control were performed in duplicate. During the first saccharification, the enzyme loading on the washed/pressed biomass was performed assuming that the glucan level was the same as the starting corn stover. However, Table 1 indicates that the glucan level was actually 6% higher on a relative basis in the wash/pressed biomass. Therefore, the enzyme loading was 18.9% in one saccharification using wash/pressed corn stover and 20% for the other using the wash/pressed corn stover. FIG. 6 illustrates the saccharification yields for both glucose and xylose. The wash/pressed samples had higher yields for both glucose and xylose. The average % increase in saccharification yield for glucose was 7.4% at 24 h and 13.9% at 48 h. The average % increase in saccharification yield for xylose was 19.2% at 24 h and 20.4% at 48 h. FIG. 6 shows that the slope of the line between the 24 h and 48 h time point is much steeper, which indicates that the saccharification of the washed/pressed biomass continues longer than for the control biomass.

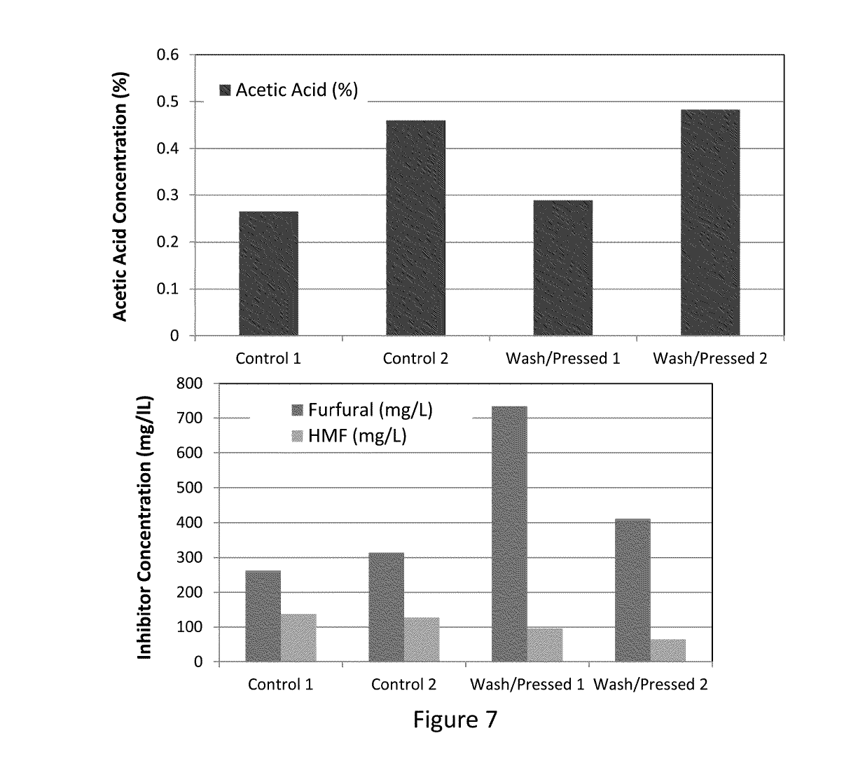

The amount of inhibitors formed during pretreatment was also measured. The acetic acid, furfural, and HMF concentrations after pretreatment are shown in FIG. 7. The acetic acid concentrations were slightly higher (between 5 and 10%) for the wash/pressed biomass. The furfural concentrations were actually considerably higher (an average of 99% increase) for the wash/pressed with an average of 573 mg/l compared to an average of 288 mg/l for the control samples. In contrast, the HMF concentrations were considerably lower (an average decrease of 39%) for the wash/pressed corn stover with an average of 80.2 mg/l compared to an average of 132 mg/l for the control samples. Without being bound by theory, it is possible that HMF formation is reduced due to the removal of the starting xylose, which would tend to convert to HMF during pretreatment. In contrast, the acetic acid and furfurals might tend to have higher concentrations because the starting carbohydrate concentration is higher after the ash is removed.

This example shows that about 91% of the glucan and 87% of the xylan were recovered in the press solids and screen solids. This number could be increased if a smaller mesh size than 250 μm were used for the screening. Additionally, these solids were enriched in carbohydrate and thus had higher saccharification efficiency than control samples. As the saccharification boost was 13.9% for glucose (over 48 h) and 20.4% for xylose (over 48 h), the sugar on a weight basis would be higher for the washed biomass even though enzyme consumption could be cut by 9%, providing cost savings.

### Example 2

This example describes a model that can be used to determine the yield and concentrations of xylose as a function of the purge ratio from a corn stover wash step.

Model Development

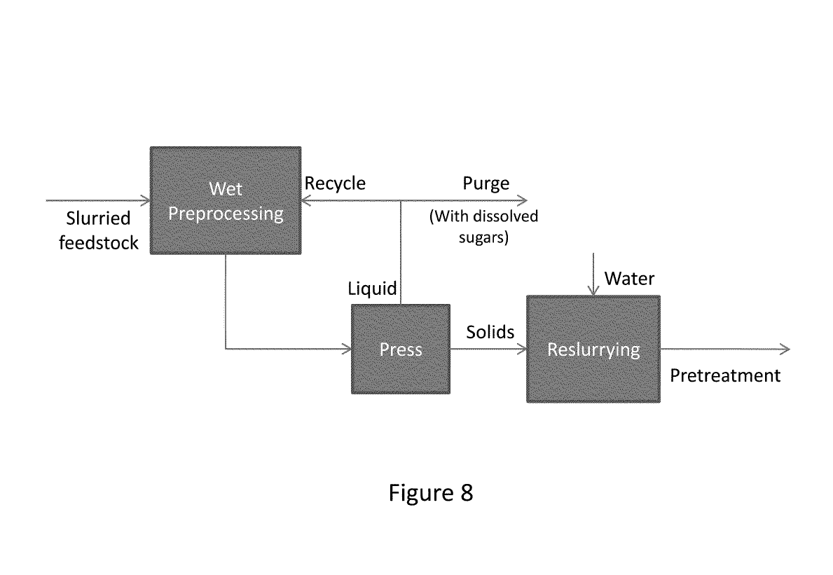

A model was developed to describe the pressing of the biomass and then recycling a fraction of the press liquor to hydrate fresh biomass (depicted in FIG. 8). The model could be programmed with the fraction of the starting biomass that is dissolved into glucose and xylose. The number fed into the model was based upon the results experimentally determined from soaking corn stover in 90° C. for 15 minutes. A value of 4.8% of the biomass was found to yield dissolved sugars. Of these sugars, 55% were glucose and 45% were xylose. The model assumed that a 15% slurry was created. Then the slurry was pressed to 45% solids with a loss of 20% of the solids in the press liquor. A fraction between 1% and 100% of the press liquor was assumed to be purged from the system. The remaining liquor was recycled back to hydrate fresh feedstock with fresh water making up the difference. The model was iterated until a steady state was reach and the value for the sugar concentration and sugar yield was determined. In this case, the 100% sugar yield was defined as the yield achieved for a 100% purge ratio. If the press performance changed (for example, less solids loss or higher solids percentage), then the predicted sugar concentrations could also change.

Results

Purge Ratio and Sugar Yield

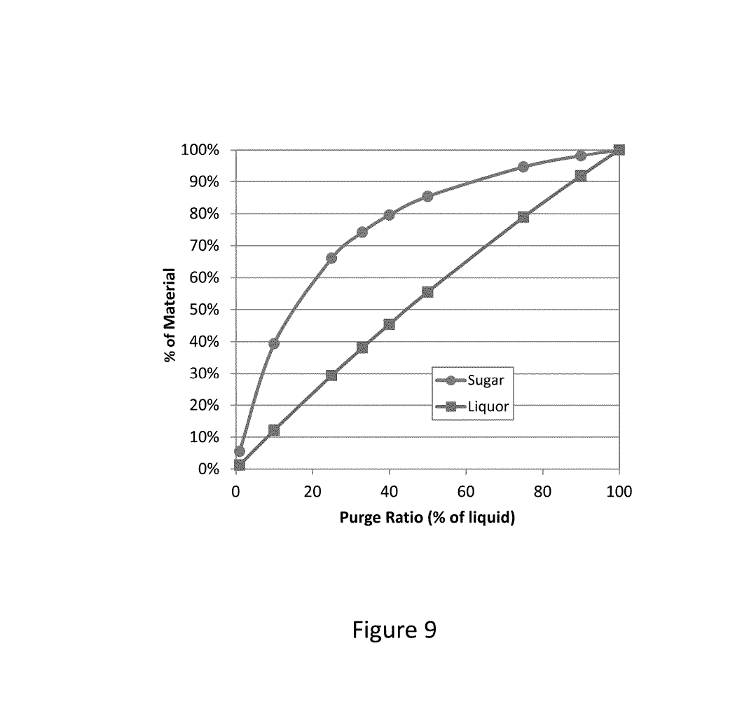

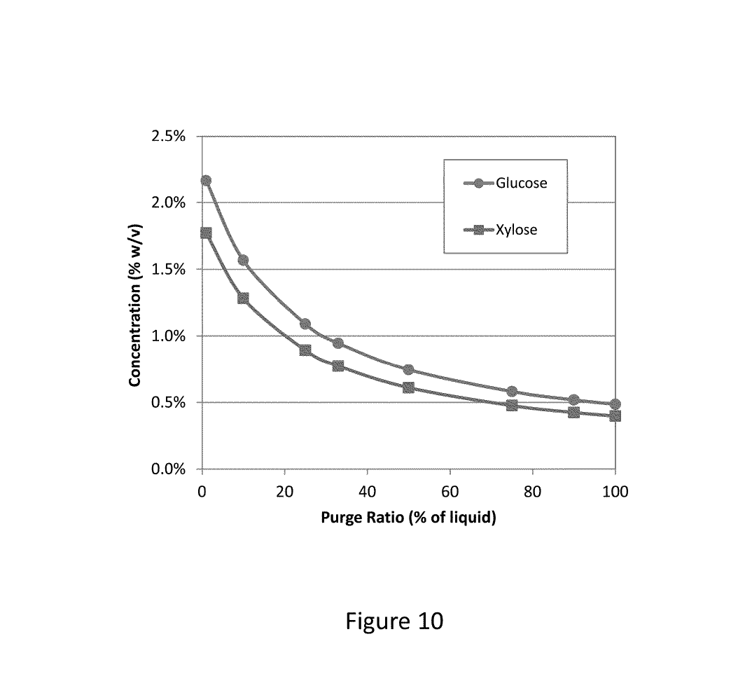

FIG. 9 illustrates the fraction of the sugar that is recovered as a function of the purge ratio. As a comparison, the fraction of the liquor that is recycled is also graphed. Because the liquor recycle rate is defined as the purge ratio, the slope of this line is 1. In contrast, the slope of the sugar line is greater than one at low purge ratios. This higher slope is because as the liquor is recycled (i.e.—lower purge ratio), the sugar concentration of the liquor increases (FIG. 10). Therefore, at low purge ratio, one obtains a relatively high sugar yield when compared to the purge ratio value.

Purge Ratio and Sugar Concentration

FIG. 10 provides a model for the glucose and xylose concentration as a function of the purge ratio. At a very low purge ratio (1%), the glucose concentration would be 2.16%, and the xylose concentration would be 1.77%. One the other extreme, for a 100% purge ratio, the glucose concentration would be 0.49%, and the xylose concentration would be 0.40%. Other levels to note are at a purge ratio of 30%, the glucose concentration in the purge has a concentration of 1.0%. At a purge ratio of 40%, the glucose concentration would be 0.85%. At a purge ratio of 45%, the glucose concentration would be 0.79%.

It should be noted that at conditions of low purge ratio, the quantity of liquid being recycled would cause the throughput in the press to be quite a bit higher than under circumstances of high purge ratios. Therefore, the advantages of higher sugar concentrations would be at least partially offset by equipment (larger pumps and presses to move and process the additional water) and the lower yields. Additionally, with lower purge ratios, there is a higher concentration of sugars in the preprocessing, which would be passed along into the pretreatment. The effect of these sugars or the use of more processing to reduce their passage into pretreatment was not evaluated in this example.

Total Sugar

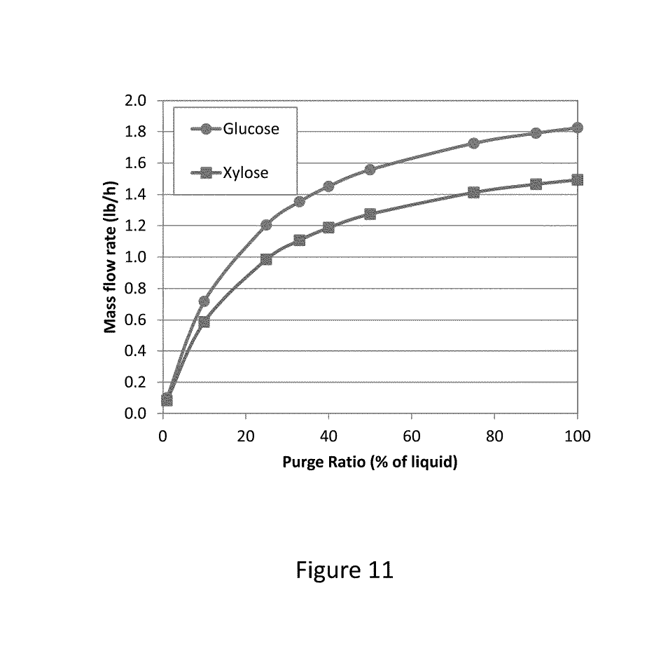

FIG. 11 displays the mass flow rate in lb/hr of the glucose and xylose as a function of the purge ratio for a 1.0 ton/day corn stover plant. These data indicate that the maximum additional sugar would be 1.8 lbs/h of glucose and 1.5 lbs/h of xylose. At a purge ratio of 33% to 45%, the glucose flow rate would be between 1.4 and 1.6 lbs/h. These numbers would scale linearly with plant throughput. Therefore, a 200 ton/day plant would have roughly 300 lbs/h of additional sugar produced through washing the biomass with a purge ratio of around 40%. At a price of $0.15 per lb of glucose, this would result in over $300,000 in revenue for the plant each year.

This example describes a model that can determine the yield and concentrations of xylose as a function of the purge ratio from a corn stover wash step. It was found that a sugar yield between 70% and 80% of the available sugars could be achieved using a liquid purge ratio of only 33% to 40%. The increased relative yield was due to a buildup of the sugars in the recycle loop. Based upon this model, a 200 ton/day plant could produce over $300,000 in additional sugar per year implementing this technology.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes. In the claims appended hereto, the term “a” or “an” is intended to mean “one or more.” The term “comprise” and variations thereof such as “comprises” and “comprising,” when preceding the recitation of a step or an element, are intended to mean that the addition of further steps or elements is optional and not excluded.

What is claimed is:

1. A method of processing biomass, comprising:
(a) contacting the biomass with a first aqueous liquid under conditions sufficient to dissolve water-soluble sugars associated with the biomass;
(b) separating the first aqueous liquid from the biomass to produce a first liquid phase comprising the water-soluble sugars and a first solids phase comprising biomass solids;
(c) contacting the first solids phase with a second aqueous liquid under conditions sufficient to render the biomass more susceptible to hydrolysis but not releasing substantial amounts of sugars, thereby producing a pretreated biomass;
(d) separating the second aqueous liquid from the pretreated biomass to produce a second liquid phase and a second solids phase; and
(e) subjecting the second solids phase to conditions sufficient to produce fermentable sugars.

2. The method of claim 1, further comprising fermenting the fermentable sugars.

3. The method of claim 1, further comprising treating the pretreated biomass to produce particles having a relatively uniform particle size.

4. The method of claim 3, wherein at least 95% of the particles have a particle size from about 5 microns to about 800 microns, or a particle size from about 5 microns to about 500 microns.

5. The method of claim 1, wherein the conditions sufficient to dissolve water-soluble sugars comprise a temperature of from about 25 to about 100 degrees C.

6. The method of claim 1, wherein the conditions sufficient to render the biomass more susceptible to hydrolysis comprise elevated temperature and pressure compared to ambient temperature and pressure.

7. The method of claim 1, wherein the subjecting comprises contacting the second solids phase with hydrolytic enzymes.

8. The method of claim 7, wherein the hydrolytic enzymes comprise one or more of cellulase, hemicellulase or an oxidoreductase such as lignin peroxidase.

9. The method of claim 1, wherein the second solids phase is mixed with at least a portion of the first liquid phase comprising the water-soluble sugars prior to or during the subjecting (e).

10. The method of claim 1, wherein at least a portion of the first liquid phase comprising the water-soluble sugars is contacted with additional biomass, and the additional biomass is processed according to the method, wherein the relative amount of the first liquid phase contacting the additional biomass is about 20% to 90%, or about 50% to 80% by weight.

11. The method of claim 1, wherein at least a portion of the first liquid phase comprising the water-soluble sugars is mixed with fermentable sugars produced by the subjecting (e).

12. The method of claim 1, wherein at least a portion of the second liquid phase is contacted with an additional solids phase prior to or during the contacting (c).

13. The method of claim 1, further comprising recycling at least a portion of the first liquid phase, at least a portion of the second liquid phase, or at least a portion of the first liquid phase and at least a portion of the second liquid phase by contacting the biomass with the at least a portion of the first liquid phase, the at least a portion of the second liquid phase, or the at least a portion of the first liquid phase and the at least a portion of the second liquid phase.

14. The method of claim 1, further comprising reducing the particle size of the biomass prior to contacting the biomass with the first aqueous liquid (a).

15. The method of claim 14, wherein at least 80% of the particles have a particle size between about 0.10 and 5.0 mm, 0.10 and 4.0 mm, 0.10 and 2.0 mm, or 0.25 to 0.85 mm.

16. The method of claim 1, wherein the solids concentration of the first solids phase after contacting with the second aqueous liquid is between about 1% and 50%, 5% and 45%, 10% and 30%, or 15% and 25% by weight.

17. The method of claim 1, wherein the solids concentration of the second solids phase that is subjected to conditions sufficient to produce fermentable sugars is at least 20%, at least 25%, at least 30%, at least 35%, at least 40%, at least 45%, or at least 50% by weight.

18. The method of claim 1, further comprising subjecting the first and/or second solids phase to one or more additional washing steps, wherein the wash liquid is added at the contacting (a) or the contacting (c).

19. The method of claim 1, wherein the first liquid phase from the separating (b) is processed to remove sand and other solid contaminants.

20. The method of claim 1, wherein the biomass from the contacting (c) is adjusted to a pH of between 3 and 6.5.

21. The method of claim 1, wherein at least a portion of the first liquid phase comprising the water-soluble sugars is concentrated and fermented.

22. The method of claim 1, wherein at least a portion of the first liquid phase comprising the water-soluble sugars is combined with corn starch and fermented.

23. The method of claim 1, wherein fermentable sugars produced by the subjecting (e) are separated from residual solids to produce a sugar syrup.

24. The method of claim 23, further comprising washing the residual solids one or more times to recover sugars.

25. The method of claim 2, wherein the fermentable sugars are separated from residual solids prior to fermentation.

26. The method of claim 1, wherein the process is a continuous process.

27. The method of claim 1, wherein the process is a batch process.

28. A processed biomass produced by the method of claim 1.
